(19) United States
(12) Patent Application Publication
(10) Pub. No.: US 2022/0370015 A1
(43) Pub. Date: Nov. 24, 2022

(54) METHODS AND SYSTEMS FOR PHOTOPLETHYSMOGRAM SIGNAL QUALITY ASSESSMENT

(71) Applicant: Analog Devices, Inc., Wilmington, MA (US)

(72) Inventors: Miles BENNETT, Stanford, CA (US); Foroohar FOROOZAN, Burlington, MA (US); Di XUE, Woburn, MA (US)

(73) Assignee: Analog Devices, Inc., Wilmington, MA (US)

(21) Appl. No.: 17/740,256

(22) Filed: May 9, 2022

Related U.S. Application Data
(60) Provisional application No. 63/186,650, filed on May 10, 2021.

(51) Int. Cl. A61B 5/00; A61B 5/024; A61B 5/0205; A61B 5/11

(52) U.S. Cl. CPC A61B 5/7221; A61B 5/7267; A61B 5/02416; A61B 5/0205; A61B 5/11

(57) ABSTRACT

Accuracy of vital signs monitoring systems depend on the quality of the measurements by the sensors. Crude techniques are applied to discard measurements which have a low signal-to-noise ratio or are saturated. A more accurate and flexible technique enables more measurements to be kept, more meaningful signal quality information to be extracted, more accurate vital signs extraction and more systems to readily embed signal quality assessment in the signal processing pipeline. Improvements include preprocessing of the signal that is independent of variations of underlying hardware, use of features with low computational complexity and high predictive power, cross-channel feature extraction, application of a trained machine learning model, and flexible translation of signal quality classification information into a continuous metric for signal quality.

### PRIORITY APPLICATION

This patent application receives benefit of and claims priority to U.S. Provisional Application, Ser. No. 63/186,650, having the same title as this patent application, filed on May 10, 2021. The US Provisional Application is incorporated by reference in its entirety.

### TECHNICAL FIELD OF THE DISCLOSURE

The present invention relates to the field of signal quality assessment, in particular to photoplethysmogram (PPG) signal quality assessment.

### BACKGROUND

Many consumer wearables and medical devices in clinical settings implement vital sign monitoring. Vital signs relate to physiological parameters of a living subject. Examples of vital signs include heart rate, heart rate variability, oxygen saturation level of the blood (SpO₂), blood pressure, body temperature, skin conductance, electrochemistry, etc. Sensors can be provided in a device to obtain measurements or signals, which can be used to infer various vital signs. Sensors can include optical sensors to make optical measurements, electrodes to make bio-potential measurements, electrodes to make bio-impedance measurements, and microelectromechanical systems (MEMS) sensors to make motion-related measurements.

One vital sign monitoring that is often implemented is heart rate monitoring. An optical system is provided to obtain and measure photoplethysmogram (PPG) signals. The optical system includes light emitting diode(s) and photodiode(s). Light emitting diode(s) illuminates a subject's skin. Photodiode(s) measure amount of light reflected off or through the subject and incident to the photodiode(s). The amount of light or light intensity changes due to dynamic blood flow occurring in the subject. The PPG signal morphology is similar to the arterial blood pressure (ABP) waveform, which makes the PPG signal suitable as a noninvasive heart rate monitoring modality. The periodicity of the PPG signal corresponds to cardiac rhythm. Therefore, heart rate and other heart-related physiological parameters can be estimated from the PPG signal. However, performance of heart rate monitoring can be degraded by poor blood perfusion, ambient light, variability in skin color, and motion artifacts.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 is an exemplary PPG system, according to some embodiments of the disclosure;

FIG. 2 is a flow diagram illustrating a method for PPG signal quality assessment and extraction of vital sign information, according to some embodiments of the disclosure;

FIG. 3 is a flow diagram illustrating an exemplary method for extraction of vital sign information, according to some embodiments of the disclosure;

FIG. 4 illustrates single-channel process for generating features, according to some embodiments of the disclosure;

FIG. 5 illustrates a multi-channel process for generating features, according to some embodiments of the disclosure;

FIG. 6 illustrates another multi-channel process for generating features, according to some embodiments of the disclosure;

FIG. 7 illustrates yet another multi-channel process for generating features, according to some embodiments of the disclosure;

FIG. 8 illustrates a process for signal quality assessment, according to some embodiments of the disclosure; and

FIG. 9 illustrates examples for implementing the process for signal quality assessment of FIG. 8, according to some embodiments of the disclosure.

### DESCRIPTION OF EXAMPLE EMBODIMENTS OF THE DISCLOSURE

Overview

Accuracy of vital signs monitoring systems depend on the quality of the measurements by the sensors. Crude techniques are applied to discard measurements which have a low signal-to-noise ratio or are saturated. A more accurate and flexible technique enables more measurements to be kept, more meaningful signal quality information to be extracted, more accurate vital signs extraction and more systems to readily embed signal quality assessment in the signal processing pipeline. Improvements include preprocessing of the signal that is independent of variations of underlying hardware, use of features with low computational complexity and high predictive power, cross-channel feature extraction, application of a trained machine learning model, and flexible translation of signal quality classification information into a continuous metric for signal quality.

Photoplethysmogram with Improved Signal Quality Assessment

Photoplethysmogram (PPG) is an optical measurement technique that detects blood volume change in an artery or vein as it moves from the heart towards the periphery of the body of a subject (e.g. fingertip, wrist, etc.). A system for making PPG measurements or obtaining PPG signals can include a light source, which illuminates the tissue of the subject, and an optical sensor that measures the intensity of the light reflected off or through the tissue. PPG signals are susceptible to various noise sources (e.g., motion), interfering signals, and other artifacts due to non-optimal placement of the optical sensor relative to the light source and/or the tissue. Any of these factors can contribute to a low-quality PPG signal, which could lead to erroneous results if the low-quality PPG signal is used as an input to a downstream algorithm.

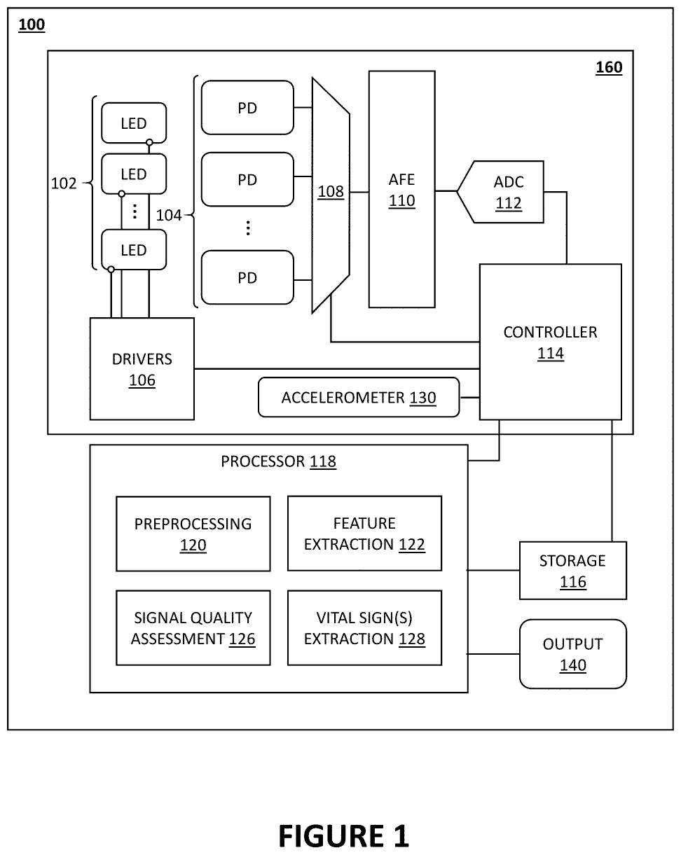

FIG. 1 is an exemplary PPG system 100, according to some embodiments of the disclosure. The PPG system 100 includes one or more light emitting diodes (LEDs) 102 as one or more light sources. A given LED may emit light having peak wavelength near or at one of the following: 460 nm (blue), 530 nm (green), 555 nm (greenish yellow), 655 nm (red), and 940 (infrared or IR). The one or more LEDs 102 may emit wavelengths. The LED(s) 102 may emit light having the same peak wavelength or different peak wavelengths. The LED(s) may emit light towards the subject, preferably the skin of the subject. In some embodiments, the PPG system 100 has a red LED, and an IR LED. In some embodiments, the PPG system has a red LED, green LED, and an IR LED. The PPG system 100 includes one or more photodiodes (PDs) 104 to sense light reflected off or through the subject. Different PPG signals can be generated by the PD(s) 104 in response to one or more selected LEDs being on. For instance, a PD can generate a red PPG signal in response to a red LED being pulsed. A PD can generate a green PPG signal in response to a green LED being pulsed. A PD can generate an IR PPG signal in response to an IR LED being pulsed.

To control the LED(s) 102 and PD(s) 104, the PPG system 100 can include a controller 114. Controller 114 can control timing of the LED(s) 102 and PD(s) 104. For instance, controller 114 can output control signals to drivers 106 (e.g., analog driver circuits) to turn selected ones of the LEDs 102 on and off. If there are multiple PDs, controller 114 can control multiplexer 108 to selectively provide a signal generated by a selected PD of the PD(s) 104 to an analog front end 110 (e.g., to amplify or filter the signal) and analog to digital converter 112 (e.g., to digitize the signal and generate digital samples). Samples of the signal from the selected PD can be stored in non-transitory computer-readable storage 116.

In some embodiments, the PPG system 100 includes a motion sensor that generates signals indicative of motion. One example of a motion sensor is an accelerometer 130. For instance, a MEMS accelerometer, e.g., a 3-axis accelerometer, may be used as accelerometer 130. Though not shown, accelerometer 130 may include analog front end circuitry (e.g., analog filter), and analog to digital converter circuitry to digitize a signal and generate digital samples to be stored in non-transitory computer-readable storage 116. A 3-axis accelerometer may generate three signals, each corresponding to motion activity along an axis.

The PPG system 100 may include a single-channel of measurement that measures a PPG signal corresponding to one of the peak wavelengths. The PPG system 100 may include multiple channels of measurement that multiple PPG signals corresponding to different peak wavelengths. The PPG system 100 may include multiple channels of measurement that measure multiple PPG signals corresponding to different peak wavelengths and one or more motion sensor signals from a motion sensor.

The PPG system 100 can include a processor 118. The processor 118 can execute instructions stored on storage 116 using data/information stored on storage 116 to carry out or execute certain processes and/or functionalities, including vital sign(s) extraction 128. To improve the accuracy of vital sign(s) extraction 128, the processor 118 can implement preprocessing 120, feature extraction 122, and signal quality assessment 126. Signal quality information generated by signal quality assessment 126 can serve as a “gate” to qualify the signal. The signal quality information can be a factor in determining whether a segment of the signal should be used or processed by vital sign(s) extraction 128, which is downstream of the signal quality assessment 126. Advantage of the gating operation can prevent low-quality signals from being used or processed by the vital sign(s) extraction 128 and thereby reduce the likelihood that erroneous or inaccurate results would be generated by the vital sign(s) extraction 128.

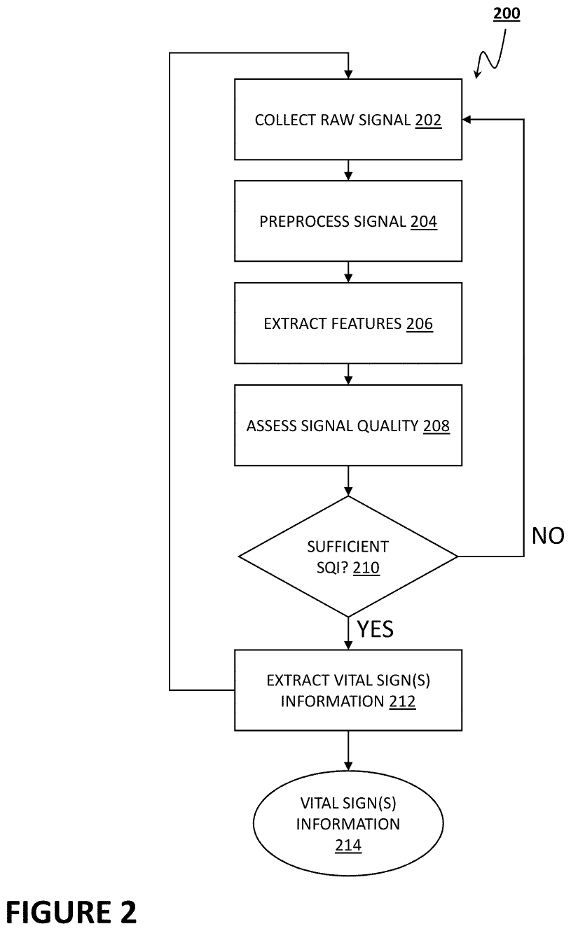

Details and advantages of such processes/functionalities for vital sign(s) extraction 128, preprocessing 120, feature extraction 122, and signal quality assessment 126, are described in FIGS. 2-9.

In some embodiments, at least a subset of the processes/functionalities can be implemented in specialized digital processing circuitry (e.g., application-specific integrated circuit) as opposed to a processor (e.g., a general purpose processor or microprocessor). For instance, any one or more of: preprocessing 120, feature extraction 122, signal quality assessment 126, and vital sign(s) extraction 128 can be implemented in PPG system 100 using digital processing circuitry. Processes/functionalities of preprocessing 120 may be particularly suitable to be implemented using specialized digital processing circuitry, which can consume less power and achieve faster processing speeds.

PPG system 100 can include output 140. In some embodiments, output 140 is to communicate vital sign information extracted by vital sign(s) extraction 128 with other device(s) or to a user. In some embodiments, output 140 is to communicate signal quality information or derivation thereof with other device(s) or to a user. Output 140 may include a display suitable to display one or more of: a numerical value, and a graphical element that indicates different numerical values. One example graphical element may include use of a color selected from a gradient of colors. Another example graphical element may include the use of different sizes of a geometrical shape (e.g., rectangular lines or bars of different length) to communicate signal quality information. Another example graphical element may include a pointer to a level within a shape (e.g., an arrow pointing to a specific point along a line or a bar) to communicate signal quality information. Another example graphical element may include a ruler, a meter, a scale, a slider, a guage, etc.

Shortcomings of Other Signal Quality Assessment Techniques

The assessment of the quality of PPG signals can be critical to the accuracy of further signal processing algorithms for vital sign(s) extraction 128 of FIG. 1. Examples of algorithms for vital sign(s) extraction include: estimating blood oxygen saturation, estimating heart rate, or predicting anomalies such as atrial fibrillation. Other approaches of assessing signal quality are often based on analyzing a signal characteristic such as the signal-to-noise ratio or the existence of signal saturation. In some other approaches, machine learning have been applied, where several signal characteristics are provided as input and the signal is classified into a discrete set of quality ratings (e.g. good/bad; excellent, acceptable, or poor; etc.). Additionally, some other approaches focus on assessing the signal quality of each PPG signal independently. The following passages describe embodiments which aim to address one or more shortcomings of such approaches.

Assessing signal quality accurately and meaningfully, and designing a practical implementation for signal quality assessment of a PPG signal are not trivial tasks. If the assessment is too conservative, many potentially usable PPG signals may be discarded. If the assessment is too crude, some potentially usable PPG signals may be discarded unnecessarily. If the assessment is not accurate, potentially usable PPG signals may be discarded, or low-quality PPG signals are passed on to the algorithm, yielding inaccurate vital signs extraction. If the assessment is too computationally complex, systems may not be able to readily embed the assessment in the signal processing pipeline to provide real time vital sign monitoring. If the assessment is too specific to the underlying sensor hardware, other systems may not be able to readily include the assessment in the signal processing pipeline. If the assessment is too specific to the vital sign extraction algorithm, other systems may not be able to readily include the assessment in the signal processing pipeline.

Method for PPG Signal Quality Assessment and Extraction of Vital Sign Information

FIG. 2 is a flow diagram illustrating a method for PPG signal quality assessment and extraction of vital sign information, according to some embodiments of the disclosure. Specifically, FIG. 2 illustrates an exemplary processes/functionalities of the PPG system 100, including processes/functionalities of circuitry 160, preprocessing 120, feature extraction 122, signal quality assessment 126, and vital sign(s) extraction 128 of FIG. 1.

In 202, raw signal is collected. The raw signal can correspond to a signal generated by a sensor, where the signal may include information relating to one or more physiological parameters of a subject. For instance, a PPG signal can be generated and collected by some of the components in circuitry 160 of FIG. 1. An accelerometer signal can be generated and collected by some of the components in circuitry 160. Raw signal can be stored in a buffer (e.g., a part of storage 116 of FIG. 1). The buffer can have a predetermined or predefined size. The predetermined or predefined size can correspond to a predetermined or predefined period of time. In some embodiments, the raw signal can be one of a red PPG signal, a green PPG signal, an infrared PPG signal, an accelerometer signal.

In 204, the raw signal is preprocessed, e.g., by preprocessing 120 of FIG. 1. For instance, the raw signal can be filtered to improve the signal in some way or make the signal more suitable for further processing downstream. In some embodiments, for the signal quality assessment algorithm to generalize across different hardware implementations or configurations (e.g., different hardware implementations or configurations of circuitry 160), several preprocessing steps can be implemented.

In some embodiments implementing 204, the raw input signal can be downsampled from its input sampling rate to a predetermined or predefined sampling rate. Input sampling rate is variable depending on the hardware implementation. Downsampling or resampling can help to fix the signal to a desired or predetermined sampling rate such that the downstream processing can be independent of the variable input sampling rate. For example, the raw input signal can be downsampled to 20 Hz, 25 Hz, or 30 Hz. The downsampled signal can be bandpass filtered to a predetermined frequency range to limit the amount of data to a specific frequency range of interest. For a given vital sign, important or salient information may reside in a limited frequency range. For instance, the downsampled signal can be bandpass filtered to a frequency range of 0.5 Hz to 10 Hz. The filtered signal can be standardized or normalized to have a value between a predetermined or predefined signal range, e.g., between 0 and 1. The process of standardization can follow this equation:

$$\tilde{x} = \frac{x - min ( x )}{max ( x ) - min ( x )}$$

In the above equation x is the input signal and \tilde{x} is the standardized signal.

In some embodiments implementing 202 and 204, the raw signal can be stored or assembled as samples into a buffer of (at least) 5.12 seconds. 5.12 seconds is a beneficial period of time due to physiology. Such a period of time can be guaranteed to include at least two complete cardiac cycles. It also turns out that 5.12 seconds of data at 25 Hz of sampling rate yields 128 samples, which can be convenient for further analysis via Fast Fourier Transform.

In 206, one or more features are extracted from the preprocessed signal, which can be stored as samples in the buffer. The samples represent a signal over a period of time. The preprocessed signal and samples are used interchangeably. In some cases, one or more features are extracted from a single preprocessed signal corresponding to a single-channel (or source of signal). In some cases, one or more features are extracted from a plurality of preprocessed signals corresponding to multiple channels (or sources of signals). Features can be derived from the statistical, spectral, and morphological properties of the signal or signal(s). An optimal set of features based on predictive power and computational complexity can be selected from Table 1 and/or Table 2. Techniques to select features with high predictive power can include: a combinatorial search, an L1 regularization path analysis, cross validation analysis, and stepwise selection. Selecting features with low computational complexity can include analyzing the processing time needed to extract a specific feature, and analyzing an amount of resources required to extract a specific feature. The relevant features can be concatenated into a vector.

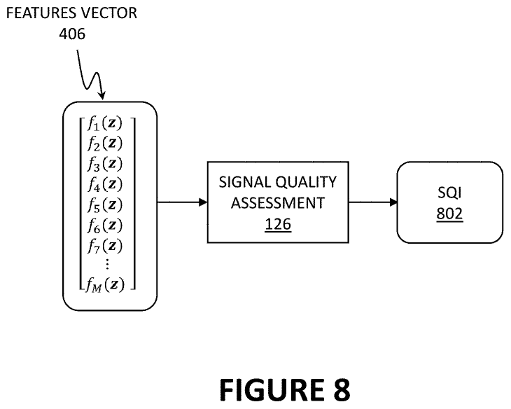

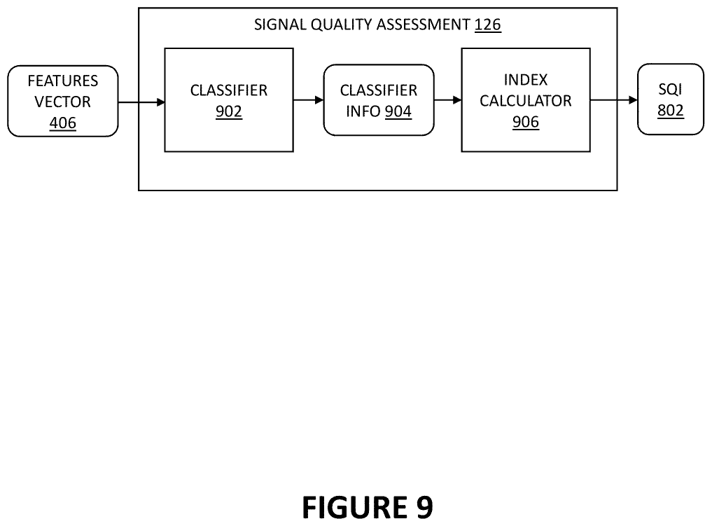

In 208, signal quality assessment is performed based on the features. A high quality signal is free of excess noise, motion artifacts, and other interfering signals. A trained machine learning model can be applied with the features as inputs. The trained machine learning model can include a classifier to generate signal quality classification information. Classes of the classifier can correspond to different discrete levels of signal quality. One exemplary set of discrete levels includes: “good” and “bad”. Another exemplary set of discrete levels includes: “excellent”, “acceptable”, and “poor”. Yet another exemplary set of discrete levels includes: “clinical grade”, “athletic grade”, “consumer grade”, and “unusable”. The signal quality classification information (e.g., discrete classes and the associated scores/probabilities) can be translated to form a signal quality index (SQI). SQI is advantageously a metric for the quality of the signal within a bounded range, e.g., 0 and 1, or 0 to 100. SQI can be a continuous metric. The translation from signal quality classification information to an SQI can differ depending on the requirements and nature of the downstream vital sign(s) extraction algorithm. The translation adds flexibility to allow for different ways to use the signal quality classification information from the trained machine learning model, which can be adapted to different vital sign(s) extraction algorithms. Details and advantages of 208 are further discussed with FIGS. 8-9.

One detailed exemplary set of discrete levels as classes for the classifier is as follows:
Excellent: The systolic and diastolic peaks are salient. The signal appears to be of “clinical quality.”
Acceptable: The systolic and diastolic peaks are not “salient,” but the heart rate can still be determined.
Poor: The signal is too noisy and heart rate cannot be determined.

In 210, a check is performed on SQI to determine if SQI is sufficient for a downstream vital sign(s) extraction algorithm. The SQI can provide an indication of signal quality. The check being performed in 210 can include a threshold. The threshold can be varied or programmable to adapt to different downstream vital sign(s) extraction algorithms. The threshold can be varied or programmable to adapt to different modes, external conditions, or requirements. The check can include determining if the SQI is above a certain threshold. The check can include determining if the SQI is above a certain threshold over time. The check can include determining if a moving average of the SQI is above a certain threshold. The check can include determining if a weighted average of the SQI is above a certain threshold.

If SQI is sufficient, the samples are used for extracting vital sign(s) information in 212. Vital sign(s) information 214 is generated as output (e.g., via output 140 of FIG. 1). If SQI is not sufficient, the samples (and potentially other samples gathered during the same time period) are not used for extracting vital sign(s) information. In some embodiments, the SQI can be used to determine whether the samples (and potentially other samples gathered during the same time period) is appropriate for further processing by downstream vital sign(s) extraction. In some embodiments, vital sign(s) extraction is not performed. In some embodiments, the samples (and potentially other samples gathered during the same time period) are discarded. Vital sign(s) information is not updated, or past/latest vital sign(s) information is maintained.

Exemplary Method for Extracting SpO₂ Information

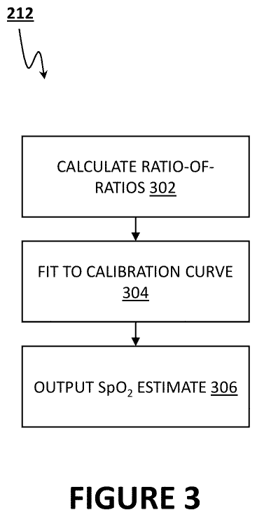

FIG. 3 is a flow diagram illustrating a method for extraction of vital sign information, according to some embodiments of the disclosure. For illustration, SpO₂ is extracted, however, it is envisioned that the various embodiments can be used with extraction of other types of vital signs (e.g., heart rate, heart rate variability, blood pressure, other heart or blood related physiological parameters).

If SQI is sufficient, samples (and potentially other samples gathered during the same time period) are used for extracting SpO₂ information. If SQI is insufficient, the samples (and potentially other samples gathered during the same time period) are discarded or not used for extracting SpO₂ information.

In 302, ratio-of-ratios is calculated, e.g., based on samples of different PPG signals (e.g., red PPG signal and IR PPG signal).

In 304, the ratio-of-ratios is fit to a calibration curve.

In 306, an SpO₂ estimate is output (e.g., via output 140 of FIG. 1).

Trained Machine Learning Model as Classifier

Referring back to FIG. 2, 208 is done by applying a trained machine learning model with features as input. The machine learning model has structures whose parameters can be trained or learned to find patterns and generate inferences, predictions, decisions, or classifications based on the model's input. Various types of machine learning models can be applied to produce signal quality classification information. For instance, a machine model can be applied to features extracted from the input signal and can output a discrete set of classes representing different signal quality levels. Examples of suitable classifiers include perceptron, naive Bayes, decision trees, (multinomial) logistic regression, k-nearest neighbor, artificial neural networks, deep learning, random forest, bagging, and adaboost. An artificial neural network can include node layers (having parameters which can be trained): an input layer to receive the features (and/or in some embodiments, the preprocessed signals), one or more hidden layers, and an output layer to produce signal quality classification information. Nodes can include its own linear regression model, which can receive an input and generate an output, based on weights and a bias. Types of artificial neural networks include feedforward neural networks, convolutional neural networks, recurrent neural networks, etc.

Preferably, the machine learning model is not computationally complex to allow for signal quality assessment to be performed in real-time with limited computing resources. Also, the machine learning model preferably has good interpretability or generates meaningful signal quality classification information to help interpret signal quality (e.g., meaningful information from which SQI can be readily calculated). Preferably, the machine learning model outputs a score or a probability for a particular features vector belonging to a particular class. The scores or probabilities corresponding to different classes can be used readily in the calculation of SQL

The machine learning model and its parameters can be trained based on labeled data. In some embodiments, the labeled data include preprocessed signals and corresponding labels. The labels can be determined based on the quality or accuracy of the resulting vital sign extracted by a given vital sign extraction algorithm based on the preprocessed signals.

For each new input (i.e., features vector) to the trained machine learning model, each feature (denoted as aₖ) can be normalized according to the following equation:

$${\tilde{a}}_{k}^{( j )} = \frac{{a}_{k}^{( j )} - {μ}_{{a}_{k}}}{{σ}_{{a}_{k}}}$$

Where:
aₖ^{(j)} is the kth feature in the features vector for an input features vector (j)
μₐ_{ₖ} is the mean of feature aₖ, calculated across the training set
σₐ_{ₖ} is the standard deviation of feature aₖ, calculated across the training set
ãₖ^{(j)} is the normalized version of aₖ^{(j)}

In some embodiments, the trained machine learning model implements a multinomial logistic classifier, and assign the signal quality classification information using the argument of the maximum (argmax) of three different binary classifiers:

z₀^{(j)}=β₀₀+Σₖ₌₁^{N}β₀ₖãₖ^{(j)}

z₁^{(j)}=β₁₀+Σₖ₌₁^{N}β₁ₖãₖ^{(j)}

z₂^{(j)}=β₂₀+Σₖ₌₁^{N}β₂ₖãₖ^{(j)}

### Where:

z₀^{(j)} is the score from the “poor” quality signal class for a given input features vector (j)
z₁^{(j)} is the score from the “acceptable” quality signal class for a given input features vector (j)
z₂^{(j)} is the score from the “excellent” quality signal class for a given input features vector (j)
ãₖ^{(j)} is the normalized kth feature of the jth input features vector
β₀ₖ is the weight associated with the “poor” quality signal class for the kth feature
β₁ₖ is the weight associated with the “acceptable” quality signal class for the kth feature
β₂ₖ is the weight associated with the “excellent” quality signal class for the kth feature
β₀₀ is the bias term associated with the “poor” quality signal class
β₁₀ is the bias term associated with the “acceptable” quality signal class
β₂₀ is the bias term associated with the “excellent” quality signal class

The trained machine learning model can output the scores corresponding to different classes as signal quality classification information.

Once the scores are calculated, the classification is the class which has the largest score:

$${y}^{( j )} = {argmax}_{i} \{ {z}_{i}^{( j )} \}$$

An alternative implementation, can provide a “probability score” for each class, which can be computed from the following set of equations:

$${p}_{0}^{( j )} = \frac{exp ( {z}_{0}^{( j )} )}{{\sum }_{i = 1}^{2} exp ( {z}_{i}^{( j )} )}$$

$${p}_{1}^{( j )} = \frac{exp ( {z}_{1}^{( j )} )}{{\sum }_{i = 1}^{2} exp ( {z}_{i}^{( j )} )}$$

$${p}_{2}^{( j )} = \frac{exp ( {z}_{2}^{( j )} )}{{\sum }_{i = 1}^{2} exp ( {z}_{i}^{( j )} )}$$

Where:
p₀^{(j)} is the probability that features vector (j) belongs to the “poor” quality signal class
p₁^{(j)} is the probability that features vector (j) belongs to the “acceptable” quality signal class
p₂^{(j)} is the probability that features vector (j) belongs to the “excellent” quality signal class

The trained machine learning model can output the probabilities corresponding to different classes as signal quality classification information.

In some embodiments, a binary logistic regression model can be implemented to readily output the signal quality classification information having probabilities associated with different classes. The probabilities can be calculated as:

$${p}^{( j )} = \frac{exp ( {β}_{0} + {\sum }_{k = 1}^{N} {β}_{k} {\tilde{a}}_{k}^{( j )} )}{1 + exp ( {β}_{0} + {\sum }_{k = 1}^{N} {β}_{k} {\tilde{a}}_{k}^{( j )} )}$$

Where:
ãₖ^{(j)} is the normalized kth feature of the jth input features vector
β₀ is the weight associated binary classifier
βₖ is the weight associated with the binary classifier for the kth feature
p^{(j)} is the probability score associated with the jth input features vector

Exemplary Single-Channel Feature Extraction Scheme

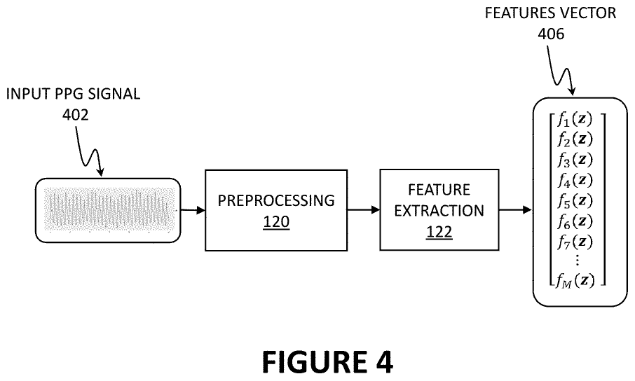

FIG. 4 illustrates single-channel process for generating features, according to some embodiments of the disclosure. An input PPG signal 402 (e.g., red PPG signal, green PPG signal, IR PPG signal) can be preprocessed according to 204 by preprocessing 120. Features can be extracted according to 206 by feature extraction 122. These features may include one or more features from in Table 1. Features are then concatenated into a features vector 406.

Exemplary Cross-Channel Feature Extraction Schemes

Some conventional systems utilizes information from different channels as a deterministic qualifier to crudely discard samples (i.e., information from different channels are not used as part of an input vector to a machine learning model). Additionally, utilizing features from a single-channel only may not always yield accurate signal quality classification information.

Some vital sign extraction techniques may rely on signals from different channels to obtain the vital sign. For instance, the oxygen saturation (SpO₂) can be estimated from the relative absorption between the red and IR PPG signals (e.g., calculating the ratio-of-ratios 302 of FIG. 3). Utilizing features from multiple channels may be beneficial for determining signal quality classification information. Signal quality classification information generated based on features from multiple channels can be more accurate for assessing whether the samples are suitable for vital sign extraction, such as SpO₂ extraction, than using single-channel features alone.

One or more features from the individual channels can be independently extracted. These features may include one or more features from in Table 1. One or more cross-channel features can be extracted from preprocessed samples from multiple channels. These cross-channel features may include one or more features in Table 2. The features from these two channels, along with cross-channel features can be concatenated into a single features vector. This features vector can then be input into a machine learning model which outputs signal quality classification information based on the received features vector.

Cross-channel features between different PPG channels may include metrics or features that indicate the signals from different channels are highly correlated or very similar. Highly correlated or very similar signals may suggest that the PPG signals are of “high quality”.

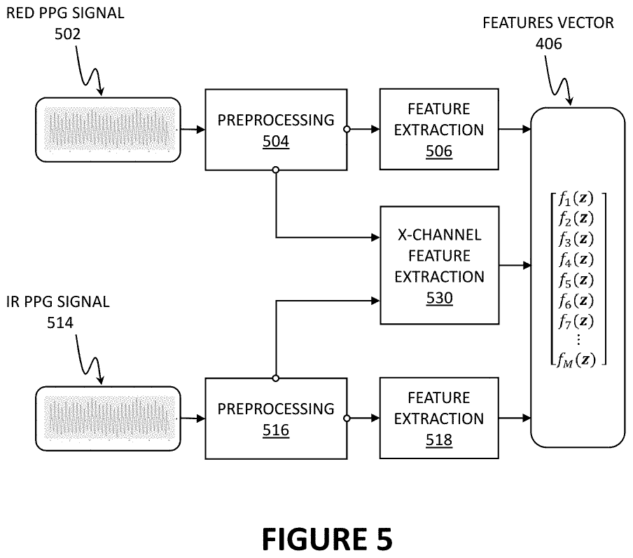

FIG. 5 illustrates a multi-channel process for generating features, according to some embodiments of the disclosure. Features vector 406 can include single-channel features and cross-channel features. In this example, two channels are provided. For illustration, the two channels include red PPG channel and an IR PPG channel. It is envisioned that other wavelength PPG channels can be provided.

A red PPG signal 502 can be preprocessed by preprocessing 504 according to 204 of FIG. 2. Feature extraction 506 can receive preprocessed red PPG signal and implement feature extraction according to 206 of FIG. 2 to extract one or more features. Preferably, two or more features are extracted for the red PPG channel. More features may increase computational complexity, but can increase accuracy of the model.

An IR PPG signal 514 can be preprocessed by preprocessing 516 according to 204 of FIG. 2. Feature extraction 518 can receive preprocessed IR PPG signal and implement feature extraction according to 206 of FIG. 2 to extract one or more features. Preferably, two or more features are extracted for the IR PPG channel. More features may increase computational complexity, but can increase accuracy of the model.

Cross-channel feature extraction 530 can receive the preprocessed red PPG signal and the preprocessed IR PPG signal from preprocessing 504 and preprocessing 516 respectively, and implement cross-channel feature extraction. Features extracted by cross-channel feature extraction 530 may include one or more features from Table 2.

Features from feature extraction 506, feature extraction 518, and cross-channel feature extraction 530 can be concatenated into a single features vector 406.

In some embodiments, the green PPG channel may have additional information about the signal quality. For instance, using one or more features from the green PPG channel may yield a more accurate estimate or extraction of a vital sign, such as heart rate, than using red and IR channels alone.

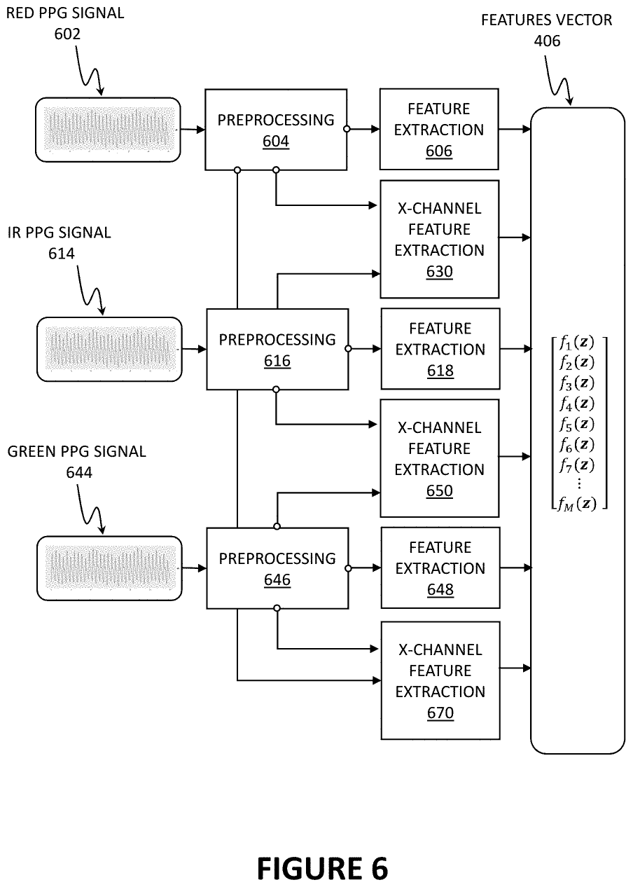

FIG. 6 illustrates another multi-channel process for generating features, according to some embodiments of the disclosure. Optionally, a green channel is added relative to FIG. 5. Additionally, optional cross-channel feature extractions are added relative to FIG. 5.

A red PPG signal 602 can be preprocessed by preprocessing 604 according to 204 of FIG. 2. Feature extraction 606 can receive preprocessed red PPG signal and implement feature extraction according to 206 of FIG. 2 to extract one or more features. Preferably, two or more features are extracted for the red PPG channel. More features may increase computational complexity, but can increase accuracy of the model.

An IR PPG signal 614 can be preprocessed by preprocessing 616 according to 204 of FIG. 2. Feature extraction 618 can receive preprocessed IR PPG signal and implement feature extraction according to 206 of FIG. 2 to extract one or more features. Preferably, two or more features are extracted for the IR PPG channel. More features may increase computational complexity, but can increase accuracy of the model.

A green PPG signal 644 can be preprocessed by preprocessing 646 according to 204 of FIG. 2. Feature extraction 648 can receive preprocessed green PPG signal and implement feature extraction according to 206 of FIG. 2 to extract one or more features. Preferably, two or more features are extracted for the green PPG channel. More features may increase computational complexity, but can increase accuracy of the model.

If desired, one or more cross-channel features can be extracted between one or more of the following pairs of channels: the red channel and IR channel, the red channel and the green channel, and the green channel and IR channel. The one or more cross-channel features can include one or more features from Table 2.

Cross-channel feature extraction 630 can receive the preprocessed red PPG signal and the preprocessed IR PPG signal from preprocessing 604 and preprocessing 616 respectively, and implement cross-channel feature extraction. Features extracted by cross-channel feature extraction 630 may include one or more features from Table 2.

Cross-channel feature extraction 650 can receive the preprocessed IR PPG signal and the preprocessed green PPG signal from preprocessing 616 and preprocessing 646 respectively, and implement cross-channel feature extraction. Features extracted by cross-channel feature extraction 650 may include one or more features from Table 2.

Cross-channel feature extraction 670 can receive the preprocessed red PPG signal and the preprocessed green PPG signal from preprocessing 604 and preprocessing 646 respectively, and implement cross-channel feature extraction. Features extracted by cross-channel feature extraction 670 may include one or more features from Table 2.

Features from one or more outputs of: feature extraction 606, feature extraction 608, feature extraction 648, cross-channel feature extraction 630, cross-channel feature extraction 650, and cross-channel feature extraction 670 can be concatenated into a single features vector 406.

In some embodiments, the motion sensor channel may have additional information about the signal quality. A three-axis accelerometer may provide additional information about the motion of the subject that may be indicative of the quality of the PPG signals. For instance, using one or more features from a motion sensor signal may yield a more accurate estimate or extraction of a vital sign, such as heart rate, than using the PPG channels alone.

Some systems may apply a crude threshold on the variance of one or more motion sensor signals corresponding to axes of the accelerometer that is used to detect dramatic motion. In some systems, an explicit correlation between one or more motion sensor signals and a PPG signal may be computed and compared against a crude threshold to determine whether to discard the PPG data. The embodiments described herein advantageously utilizes information from the motion sensor signal(s) as one or more features as input to the machine learning model. Using information from the motion sensor signal(s) as a feature has the added benefit of being more accurate by detecting interactions among features from other channels. Such (hidden) interactions would be detected by the machine learning model, whereas a crude threshold on the variance or correlation would not be able to detect such interactions.

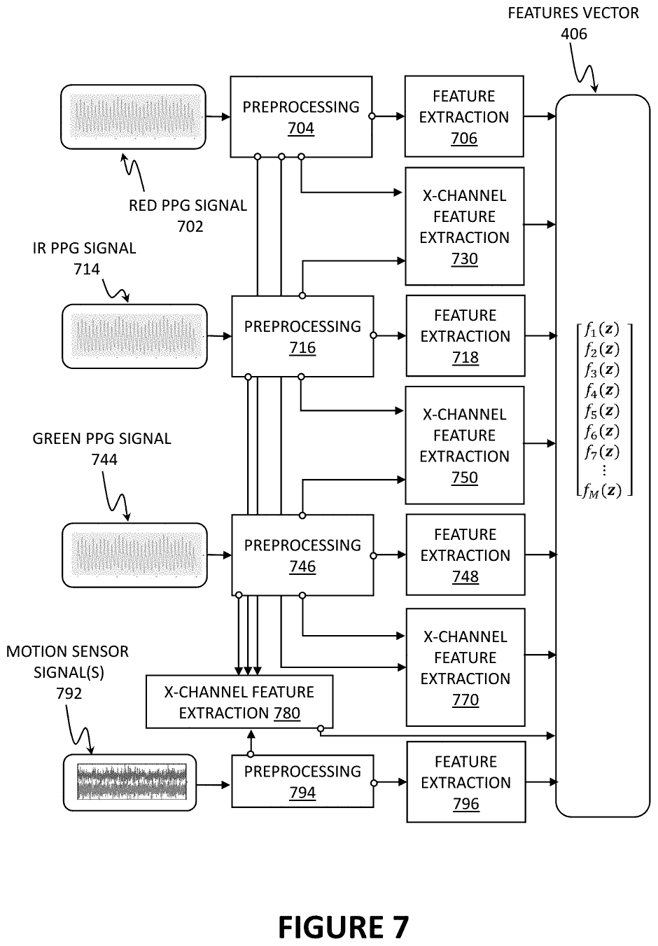

FIG. 7 illustrates yet another multi-channel process for generating features, according to some embodiments of the disclosure. The features from the red, green, and IR PPG signals are independently extracted in addition to features from accelerometer. Relative to FIG. 6, a motion sensor channel and optional cross-channel feature extractions between the motion sensor channel and PPG channels are added.

A red PPG signal 702 can be preprocessed by preprocessing 704 according to 204 of FIG. 2. Feature extraction 706 can receive preprocessed red PPG signal and implement feature extraction according to 206 of FIG. 2 to extract one or more features. Preferably, two or more features are extracted for the red PPG channel. More features may increase computational complexity, but can increase accuracy of the model.

An IR PPG signal 714 can be preprocessed by preprocessing 716 according to 204 of FIG. 2. Feature extraction 718 can receive preprocessed IR PPG signal and implement feature extraction according to 206 of FIG. 2 to extract one or more features. Preferably, two or more features are extracted for the IR PPG channel. More features may increase computational complexity, but can increase accuracy of the model.

A green PPG signal 744 can be preprocessed by preprocessing 746 according to 204 of FIG. 2. Feature extraction 748 can receive preprocessed green PPG signal and implement feature extraction according to 206 of FIG. 2 to extract one or more features. Preferably, two or more features are extracted for the green PPG channel. More features may increase computational complexity, but can increase accuracy of the model.

One or more motion sensor signals 792 can be preprocessed by preprocessing 794 according to 204 of FIG. 2. Feature extraction 796 can receive preprocessed accelerometer signal and implement feature extraction according to 206 of FIG. 2 to extract one or more features. Preferably, two or more features are extracted for the motion sensor channel. More features may increase computational complexity, but can increase accuracy of the model. The features may be computed independently for each axis of a motion sensor. Alternatively, the features may be computed based on the magnitude of the acceleration: aᵣ=\sqrt{aₓ²+a_{y}²+a_{z}²}. In some embodiments, the cross-channel features are extracted between a PPG signal and a motion sensor signal correspond to a single axis, between a PPG signal and a magnitude of the acceleration combining two axes, or between a PPG signal and a magnitude of the acceleration combining all three axes. Exemplary features for the motion sensor signal(s) includes one or more features from Table 1. In some embodiments, features extracted includes: variance, skewness, and kurtosis (see details in Table 1). Such features can be beneficial for detecting the presence of dramatic motion.

If desired, one or more cross-channel features can be extracted between one or more of the following pairs of channels: the red channel and IR channel, the red channel and the green channel, the green channel and IR channel, the red channel and the motion sensor channel, the IR channel and the motion sensor channel, and the green channel and the motion sensor channel. The one or more cross-channel features can include one or more features from Table 2.

When crossing between a PPG channel and a motion sensor channel, the cross-channel features preferably indicate a degree of similarity (dissimilarity) or correlation (independence) between the motion sensor signal and a PPG signal. Dissimilarity or independence can suggest that the PPG signals are of “high quality”. A high degree of similarity or correlation between the motion sensor signal and the PPG signal suggests that the PPG signals are of “low-quality”, potentially due to the presence of a motion artifact or a hardware malfunction.

Cross-channel feature extraction 730 can receive the preprocessed red PPG signal and the preprocessed IR PPG signal from preprocessing 704 and preprocessing 716 respectively, and implement cross-channel feature extraction. Features extracted by cross-channel feature extraction 730 may include one or more features from Table 2.

Cross-channel feature extraction 750 can receive the preprocessed IR PPG signal and the preprocessed green PPG signal from preprocessing 716 and preprocessing 746 respectively, and implement cross-channel feature extraction. Features extracted by cross-channel feature extraction 750 may include one or more features from Table 2.

Cross-channel feature extraction 770 can receive the preprocessed red PPG signal and the preprocessed green PPG signal from preprocessing 704 and preprocessing 746 respectively, and implement cross-channel feature extraction. Features extracted by cross-channel feature extraction 770 may include one or more features from Table 2.

Cross-channel feature extraction 780 can receive the preprocessed motion sensor signal(s) from preprocessing 794 and one of: the preprocessed red PPG signal from preprocessing 704, the preprocessed IR PPG signal from preprocessing 716, and the preprocessed green PPG signal from preprocessing 746. Cross-channel feature extraction 780 can implement cross-channel feature extraction. Features extracted by cross-channel feature extraction 780 may include one or more features from Table 2. In some embodiments, cross-channel feature extraction may be implemented as different instances to perform different pairwise cross-channel feature extraction between the preprocessed motion sensor signal(s) and one of: the preprocessed red PPG signal from preprocessing 704, the preprocessed IR PPG signal from preprocessing 716, and the preprocessed green PPG signal from preprocessing 746.

Features from one or more outputs of: feature extraction 706, feature extraction 718, feature extraction 748, feature extraction 796, cross-channel feature extraction 730, cross-channel feature extraction 750, cross-channel feature extraction 770, and cross-channel feature extraction 780 (or different instances thereof) can be concatenated into a single features vector 406.

Signal Quality Assessment and Calculation of SQI

FIG. 8 illustrates a process for signal quality assessment, according to some embodiments of the disclosure. Features vector 406 is provided as input to signal quality assessment 126, which implements 208 of FIG. 2. Signal quality assessment 126 generates SQI 802 as output.

FIG. 9 illustrates examples for implementing the process for signal quality assessment of FIG. 8, according to some embodiments of the disclosure. Features vector 406 is provided as input to a classifier 902 to generate signal quality classification information 904. Details relating to the classifier 902 are mentioned herein, e.g., under section titled “Trained machine learning model as classifier”. Signal quality classification information 904 is then used by index calculator 906 as input to calculate SQI 802.

There are several techniques by which the signal quality classification information 904 output from the classifier 902 can be used by index calculator 906 to compute an SQL Exemplary techniques include: SQI based on classification, SQI based on probability of being of the highest quality class, and SQI based on a weighted sum. The technique implemented in index calculator 906 is flexible and can be adapted to the specific downstream vital sign(s) extraction algorithm.

To determine SQI based on classification, SQI can be computed from the classification y^{(j)} (i.e., index i for class with the highest score or probability or highest confidence or match), and then computes the SQI as:

$${SQI}^{( j )} = \frac{{y}^{( j )}}{2}$$

From the above equation, an “excellent” quality signal can be assigned a value of 1, an “acceptable quality signal” can be assigned a value of 0.5, and a “poor” quality signal can be assigned a value of 0. In other words, the value of SQI is assigned based on the classification with the highest score or probability.

To determine SQI based on probability of the highest signal quality class, SQI can be calculated by using the probability of the input signal being of the “excellent” quality signal class:

SQI^{(j)}=p₂^{(j)}

To calculate SQI with a weighted sum, the SQI can be calculated based on a weight sum of each of the probability scores:

SQI^{(j)}=c₀p₀^{(j)}+c₁p₁^{(j)}+c₂p₂^{(j)}

Where:
c₀ is the weight associated with the “poor” quality class (an exemplary value can be c₀=0)
c₁ is the weight associated with the “acceptable” quality class (an exemplary value can be c₁=0.5)
c₂ is the weight associated with the “excellent” quality class (an exemplary value can be c₂=1.0)

### VARIATIONS AND IMPLEMENTATIONS

Moreover, certain embodiments discussed above can be provisioned in devices and systems for medical imaging, patient monitoring in a clinical setting, medical instrumentation, and home healthcare monitoring. Other devices and systems that can benefit from the embodiments described herein include consumer electronics, sports/athlete electronics, animal/pet/livestock monitoring systems, and baby monitoring systems. The embodiments described herein can also be beneficial to other applications having a PPG system, or any suitable physiological parameter monitoring system. In some scenarios, the embodiments described herein can be applied to machine monitoring.

In the discussions of the embodiments above, various electrical components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure.

Parts of various circuitry for signal quality assessment can include electronic circuitry to perform the functions described herein. In some cases, one or more parts of the circuitry can be provided by a processor specially configured for carrying out the functions described herein. For instance, the processor may include one or more application-specific components, or may include programmable logic gates which are configured to carry out the functions describe herein. The circuitry can operate in analog domain, digital domain, or in a mixed signal domain. In some instances, the processor may be configured to carrying out the functions described herein by executing one or more instructions stored on a non-transitory computer-readable medium using and operating on data/information stored on the non-transitory computer-readable medium. In some embodiments, an apparatus can include means for performing or implementing one or more of the functionalities describe herein.

It is also imperative to note that all of the specifications, dimensions, and relationships outlined herein (e.g., the number of processors, logic operations, etc.) have only been offered for purposes of example and teaching only. Such information may be varied considerably without departing from the spirit of the present disclosure. The specifications apply only to one non-limiting example and, accordingly, they should be construed as such. In the foregoing description, example embodiments have been described with reference to particular processor and/or component arrangements. Various modifications and changes may be made to such embodiments without departing from the scope of the disclosure. The description and drawings are, accordingly, to be regarded in an illustrative rather than in a restrictive sense.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in this Specification, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It is also important to note that the functions related to signal quality assessment, illustrate only some of the possible functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure.

### SELECT EXAMPLES

Example 1001. A method for signal quality assessment that is independent of variations of underlying hardware to generate a sensor signal, comprising: downsampling a sensor signal to a predefined sampling rate; bandpass filtering the downsampled sensor signal; standardizing the filtered signal to be between a predefined signal range; extracting features from the standardized signal; and receiving features and outputting signal quality classification information based on the received features.

Example 1001. The method of Example 1001, wherein outputting the signal quality classification information comprises transforming the signal quality classification information into a signal quality index.

Example 1003. The method of Example 1001 or 1002, wherein the signal quality classification information comprises a signal quality index.

Example 2001. A method for signal quality assessment, comprising: extracting first features from samples representative of a first photoplethysmogram signal over a period of time, wherein the first features include two or more of features shown in Table 1; receiving, by a trained machine learning model, first features and outputting signal quality classification information based on the received first features; and determining a signal quality assessment based on the signal quality classification information.

Example 2002. The method of Example 2001, further comprising: extracting second features from samples representative of a second photoplethysmogram signal over a period of time; wherein: the second features include two or more of features shown in Table 1; and the trained machine learning model further receives the second features and outputs the signal quality classification information further based on the received second features.

Example 2003. The method of Example 2001 or 2002, further comprising: extracting third features from samples representative of a motion sensor signal over a period of time; wherein the trained machine learning model further receives the third features and outputs the signal quality classification information further based on the received third features.

Example 2004. The method of one of Examples 2001-2003, further comprising: extracting one or more first cross-channel features from the samples representative of the first photoplethysmogram signal over the period of time, and samples representative of a second photoplethysmogram signal over the period of time; wherein: the one or more first features include one or more of features shown in Table 2; and the trained machine learning model further receives the first cross-channel features and outputs the signal quality classification information further based on the received first cross-channel features.

Example 2005. The method of one of Examples 2001-2004, further comprising: extracting one or more second cross-channel features from the samples representative of the first photoplethysmogram signal over the period of time, and samples representative of a motion sensor signal over the period of time; wherein the trained machine learning model further receives the second cross-channel features and outputs the signal quality classification information further based on the received second cross-channel features.

Example 3001. A vital sign monitoring device, comprising: a buffer to store samples representative of a photoplethysmogram signals over a period of time that includes at least two cardiac cycles; processor, when executing a set of instructions stored on non-transitory computer-readable medium, to: process the samples; extract features from the preprocessed samples; apply a trained machine learning model using the features as input; generate signal quality classification information based on the machine learning model; extract vital sign information based on the samples if the signal quality classification indicates sufficient quality; and not extract vital sign information based on the samples if the signal quality classification does not indicate sufficient quality; and user interface output to output extracted vital sign information to a user.

Example 4001. A vital sign monitoring device, comprising: a buffer to store samples representative of a photoplethysmogram signals over a period of time that includes at least two cardiac cycles; circuitry to preprocess the samples; processor, when executing a set of instructions stored on non-transitory computer-readable medium, to: extract features from the preprocessed samples; apply a trained machine learning model using the features as input; and generate signal quality classification information based on the machine learning model; extract vital sign information based on the samples if the signal quality classification indicates sufficient quality; and not extract vital sign information based on the samples if the signal quality classification does not indicate sufficient quality; and user interface output to output extracted vital sign information to a user.

Example 5001. A method for signal quality assessment, comprising: extracting features from samples representative of a photoplethysmogram signal over a period of time; receiving, by a trained machine learning model, features and outputting signal quality classification information based on the received features; and transforming the signal quality classification information to a signal quality index.

Example 5002. The method of Example 5001, wherein the signal quality classification information includes a score associated with each classification.

Example 5003. The method of Example 5001 or 5002, wherein the signal quality classification information includes a probability associated with each classification.

Example 5004. The method of one of Examples 5001-5003, wherein the signal quality index is a value within a bounded range.

Example 5005. The method of one of Examples 5001-5004, wherein the signal quality index is a continuous metric within a bounded range.

Example 5006. The method of one of Example 5001-5005, wherein transforming the signal quality classification information to the signal quality index comprises: assigning a value as a signal quality index based on the classification with a highest score.

Example 5007. The method of one of Example 5001-5006, wherein transforming the signal quality classification information to the signal quality index comprises: assigning a probability as a signal quality index, wherein the probability is associated with one of classes in the signal quality classification information.

Example 5008. The method of one of Example 5001-5007, wherein transforming the signal quality classification information to the signal quality index comprises: assigning a weighted sum of probabilities as a signal quality index, wherein the probabilities are associated with classes in the signal quality classification information.

Example 5009. The method of one of Example 5001-5008, wherein the trained machine learning model comprises a neural network based model.

Example 6001. A method for signal quality assessment, comprising: extracting first features from first samples representative of a first photoplethysmogram signal over a period of time, wherein the first features include two or more of features shown in Table 1; extracting second features from second samples representative of a second photoplethysmogram signal over the period of time, wherein the second features include two or more of features shown in Table 1; extracting one or more cross-channel features from the first and second samples; receiving, by a trained machine learning model, the first features, the second features, and sample correlation coefficient, and outputting signal quality classification information based on the received features; and generating a signal quality assessment based on the signal quality classification information.

Example 6002. The method of Example 6001, wherein the first photoplethysmogram signal is a red photo-plethysmogram signal, and the second photo-plethysmogram signal is an infrared photo-pletheysmogram signal.

Example 6003. The method of Example 6001, wherein the first photoplethysmogram signal is a red photoplethysmogram signal, and the second photoplethysmogram signal is a green photoplethysmogram signal.

Example 6004. The method of Example 6001, wherein the first photoplethysmogram signal is a green photoplethysmogram signal, and the second photoplethysmogram signal is an infrared photoplethysmogram signal.

Example 6005. The method of one or more of Examples 6001-6004, further comprising: extracting fourth feature(s) from third samples representative of a motion sensor signal over the period of time, wherein the fourth feature(s) includes one or more of: variance, skewness, and kurtosis; and receiving, by the trained machine learning model, the fourth feature(s).

Example 6006. The method of one or more of Examples 6001-6005, wherein the one or more cross-channel features include: one or more of features shown in Table 2.

Example 6007. The method of one or more of Examples 6001-6006, wherein the one or more cross-channel features indicate a degree of similarity between the first and second samples.

Example 7001. A method for signal quality assessment, comprising: extracting first features from first samples representative of a photoplethysmogram signal over a period of time, wherein the first features include two or more of features shown in Table 1; extracting second feature(s) from second samples representative of a motion sensor signal over the period of time; extracting one or more cross-channel features from the first and second samples; receiving, by a trained machine learning model, the first features, second features, and sample correlation coefficient, and outputting signal quality classification information based on the received features; and generating a signal quality assessment based on the signal quality classification information.

Example 7002. The method of Example 7001, wherein the one or more cross-channel features indicate a degree of dissimilarity between the first and second samples.

Example 7003. The method of Example 7001 or 7002, wherein the one or more cross-channel features include one or more features from Table 2.

Example 7004. The method of one of Examples 7001-7003, wherein the second feature(s) includes one or more of: variance, skewness, and kurtosis.

TABLE 1
Exemplary single-channel features as part of features vector 406

| Single-Channel | |
|---|---|
| Feature | Illustrative Implementations |
| Number of zero | A count of zero crossings, or a number of times the signal crosses |
| crossings | zero, or changes polarity/sign |
| Elgendi signal-to-noise | Elgendi SNR is a measure of SNR developed by Mohamed Elgendi to |
| ratio (SNR) | characterize PPG signals. A input signal x[n] is filtered with a |
| | bandpass filter, e.g., having a pass frequency range of 0.4 Hz ≤ f ≤ |
| | 5 Hz and generates an output signal y[n]. Elgendi SNR can be |
| | obtained by: |
| | ${SNR}_{E} = \frac{Var ( \vert Y \vert )}{Var ( Y )}$ |
| | Where: |
| | \|Y\| represents the absolute value of the signal |
| Hjorth mobility | Hjorth mobility measure the mean frequency of the standard |
| | deviation of the power spectrum of the signal; however, it can be |
| | computed in the time domain. |
| | ${Hjorth}_{M} ( X ) = \sqrt{\frac{Var [ {X}^{′} ]}{Var [ X ]}}$ |
| | In the above equation, X′ is the derivative (or as best can be |
| | approximated in discrete time) of the input signal X, which can be |
| | calculated as: |
| | x′[n] = x[n] − x[n − 1] |
| Hjorth complexity | Hjorth complexity is a measure for the bandwidth of the signal. |
| | ${Hjorth}_{C} ( X ) = \frac{{Hjorth}_{M} ( {X}^{′} )}{{Hjorth}_{M} ( X )}$ |
| Perfusion (also | Perfusion is the pulsatile component of the blood to the static or |
| referred to as | DC-component of the blood. Modulation index is a way of |
| modulation index) | measuring perfusion index optically using PPG |
| Shannon entropy | A measure of information in the samples |
| Autocorrelation peaks | A measure of relative height of the 2^{nd}, 3^{rd}, 4^{th}, etc. peaks in the |
| | autocorrelation as compared to the variance of the signal |
| Sample entropy | A measure of complexity in the signal |
| Relative power | The energy of the signals after bandpass filtering relative to the |
| | energy of the raw PPG signals |
| Skewness | Skewness is a measure of the asymmetry of the probability |
| | distribution. A normal distribution should have skewness of 0. For a |
| | given signal X, the skewness can be calculated as: |
| | $Skew ( X ) = \frac{\frac{1}{N} {\sum }_{n = 0}^{N - 1} {( x [ n ] - {μ}_{x} )}^{3}}{{( \frac{1}{N} {\sum }_{n = 0}^{N - 1} {( x [ n ] - {μ}_{x} )}^{2} )}^{3 / 2}}$ |
| Skewness of the | PSD may be computed using a variety of methods, including |
| power spectral density | Barlett's method, Welch's method, and periodogram |
| (PSD) | Skewness can be obtained from the PSD |
| Kurtosis | Kurtosis is a measure of how pronounced the ″tails″ of a |
| | distribution are. High kurtosis generally means there is large |
| | number of outliers. For a given signal X, the kurtosis can be |
| | calculated as: |
| | $Kurt ( X ) = \frac{\frac{1}{N} {\sum }_{n = 0}^{N - 1} {( x [ n ] - {μ}_{x} )}^{4}}{{( \frac{1}{N} {\sum }_{n = 0}^{N - 1} {( x [ n ] - {μ}_{x} )}^{2} )}^{2}}$ |
| Kurtosis deviation | A measure of Kurtosis relative to the Kurtosis of an ideal sine wave |
| Kurtosis of the power | PSD may be computed using a variety of methods, including |
| spectral density (PSD) | Barlett′s method, Welch′s method, and periodogram |
| | Kurtosis can be obtained based on the PSD |
| Variance | Variance measures how far ″spread out″ a probability distribution |
| | is. From a signal processing perspective, variance is one measure for |
| | average power of signal. |
| | $Var ( x ) = \frac{1}{N} {\sum }_{n = 0}^{N - 1} {( x [ n ] - {μ}_{x} )}^{2}$ |
| | Where: |
| | μₓ is the mean of the signal x[n] |
| Relative energy in | PSD may be computed using a variety of methods, including |
| various power spectral | Barlett′s method, Welch′s method, and periodogram |
| density (PSD) bands | Energy in the PSD bands can be computed relative to the overall |
| | energy of the signal |
| Relative peak heights | PSD may be computed using a variety of methods, including |
| in the power spectral | Barlett′s method, Welch′s method, and periodogram |
| density (PSD) | Find the three highest peaks in the PSD (using a suitable peak |
| | finding method) |
| | Calculate ratio of the 2^{nd} highest peak relative to the 1^{st} highest |
| | peak and ratio of the 3^{rd} highest peak relative to the 1^{st} highest peak |
| | ${r}_{1 , 2} = \frac{{p}_{2}}{{p}_{1}}$ |
| | Peak can be magnitude or energy |
| Relative peak | PSD may be computed using a variety of methods, including |
| distances in the power | Barlett's method, Welch's method, and periodogram |
| spectral density (PSD) | Find the three highest peaks in the PSD (using a suitable peak |
| | finding method) |
| | Record relative locations (in frequency) for the three highest peaks |
| | relative |
| | d_{1,2} = f₂ − f₁ |
| | d_{2,3} = f₃ − f₂ |
| | f₁ is the location of the 1^{st} highest peak |
| | f₂ is the location of the 2^{nd} highest peak |
| | f₃ is the location of the 3^{rd} highest peak |
| | Peak can be magnitude or energy |
| Peak height standard | This feature can be calculated based on the standard deviation of |
| deviation | the height of each of the prominent peaks (local maxima) in the |
| | samples |
| | In order to remove high frequency noise, the samples can be low- |
| | pass filtered or bandpass filtered |
| Peak trough standard | This feature can be calculated based on the standard deviation of |
| deviation | the depth of each of the prominent troughs (local minima) in the |
| | samples |
| | In order to remove high frequency noise, the samples can be low- |
| | pass filtered or bandpass filtered |
| Peak height mean | This feature is calculated based on the mean height of each of the |
| | prominent peaks (local maxima) in the samples. |
| | In order to remove high frequency noise, the samples can be low- |
| | pass filtered or bandpass filtered |
| Peak trough mean | This feature is calculated based on the mean depth of each of the |
| | prominent troughs (local minima) in the samples. |
| | In order to remove high frequency noise, the samples can be low- |
| | pass filtered or bandpass filtered |
| Mean peak-to-peak | A number of peaks can be identified in the samples. |
| time difference | In order to remove high frequency noise, the samples can be low- |
| | pass filtered or bandpass filtered |
| | Time differences between peaks can be recorded. A mean can be |
| | calculated based on recorded time differences |
| Standard deviation | A number of peaks can be identified in the samples. |
| peak-to-peak time | In order to remove high frequency noise, the samples can be low- |
| difference | pass filtered or bandpass filtered |
| | Time differences between peaks can be recorded. Standard |
| | deviation can be calculated based on recorded time differences |
| Trough depth mean | This feature is calculated based on the mean depth of each of the |
| | prominent troughs (local minima) in the input signal X. In order to |
| | remove high frequency noise, the input signal is typically low-pass |
| | filtered or bandpass filtered |
| Trough depth standard | This feature is calculated based on the standard deviation depth of |
| deviation | each of the prominent troughs (local minima) in the input signal X. |
| | In order to remove high frequency noise, the input signal is typically |
| | low-pass filtered or bandpass filtered |
| Mean trough-to- | A number of troughs can be identified in the samples. |
| trough time difference | In order to remove high frequency noise, the samples can be low- |
| | pass filtered or bandpass filtered |
| | Time differences between troughs can be recorded. A mean can be |
| | calculated based on recorded time differences |
| Standard deviation | A number of troughs can be identified in the samples. |
| trough-to-trough time | In order to remove high frequency noise, the samples can be low- |
| difference | pass filtered or bandpass filtered |
| | Time differences between troughs can be recorded. Standard |
| | deviation can be calculated based on recorded time differences |
| Template matching | This feature measures average distance of all of the extracted |
| with median pulse via | pulses from the median pulse, where distance is the Euclidian |
| dynamic time warping | distance calculated after dynamic time warping |
| Absolute Value signal- | One measure of SNR can be easily computed is based on the |
| to-noise ratio (SNR) | variance of the absolute value of the signal X: |
| | ${SNR}_{A} = \frac{Var ( \vert X \vert )}{Var ( X )}$ |
| | In order to remove a DC offset, the X may be first high pass filtered |

TABLE 2
Exemplary cross-channel features as part of features vector 406

| Cross-Channel Feature | Illustrative Implementations |
|---|---|
| Cosine similarity | A measure of similarity between two non-zero vectors of an inner |
| | product space |
| | Can be computed based on samples from different channels, using |
| | the samples as the two vectors |
| Cross-entropy | A measure of cross-entropy between two probability distributions |
| | Can be computed based on samples from different channels |
| | modeled as two probability distributions |
| Normalized maximum | The maximum of the cross-correlation of two signals x and y (e.g. |
| of the cross- | the red PPG signal and the IR PPG signal) divided by the standard |
| correlation | deviation of x and the standard deviation of y |
| Wasserstein distance | A measure of distance between two probability distributions on a |
| | given metric space. |
| | Can be computed based on samples from different channels |
| | modeled as two probability distributions |
| Earth Mover′s distance | A measure of distance between two probability distributions over a |
| | region. |
| | Can be computed based on samples from different channels |
| | modeled as two probability distributions |
| Mutual information | A measure of mutual dependence between two variables |
| | Can be computed based on samples from different channels |
| | modeled as two variables |
| Sample correlation coefficient | ${r}_{xy} = \frac{{\sum }_{n = 1}^{N} ( x [ n ] - {μ}_{x} ) ( y [ n ] - {μ}_{y} )}{\sqrt{{\sum }_{n = 1}^{N} {( x [ n ] - {μ}_{x} )}^{2}} \sqrt{{\sum }_{n = 1}^{N}} {( y [ n ] - {μ}_{y} )}^{2}}$ |
| | Where: |
| | x and y are two signals (e.g. PPG signal from the red LED, |
| | PPG signal from the green LED, etc.) |
| | μₓ is the mean of x |
| | μ_{y} is the mean of y |

What is claimed is:

1. A method for signal quality assessment, comprising:
extracting first features from samples representative of a first photoplethysmogram signal over a period of time;
receiving, by a machine learning model, first features and outputting signal quality classification information based on the received first features; and
transforming the signal quality classification information to a signal quality index.

2. The method of claim 1, further comprising:
extracting second features from samples representative of a second photoplethysmogram signal over a period of time; and
receiving, by the machine learning model, the second features and outputting the signal quality classification information further based on the received second features.

3. The method of claim 1, further comprising:
extracting third features from samples representative of a motion sensor signal over a period of time; and
receiving, by the machine learning model, the third features and outputting the signal quality classification information further based on the received third features.

4. The method of claim 1 further comprising:
extracting one or more first cross-channel features from the samples representative of the first photoplethysmogram signal over the period of time, and samples representative of a second photoplethysmogram signal over the period of time; and
receiving, by the machine learning model, the first cross-channel features and outputting the signal quality classification information further based on the received first cross-channel features.

5. The method of claim 1, further comprising:
extracting one or more second cross-channel features from the samples representative of the first photoplethysmogram signal over the period of time, and samples representative of a motion sensor signal over the period of time; and
receiving, by the machine learning model, the second cross-channel features and outputting the signal quality classification information further based on the received second cross-channel features.

6. The method of claim 1, wherein the signal quality classification information includes a score associated with each classification.

7. The method of claim 1, wherein the signal quality classification information includes a probability associated with each classification.

8. The method of claim 1, wherein the signal quality index is a value within a bounded range.

9. The method of claim 1, wherein the signal quality index is a continuous metric within a bounded range.

10. The method of claim 1, wherein transforming the signal quality classification information to the signal quality index comprises:
assigning a value as a signal quality index based on the classification with a highest score.

11. The method of claim 1, wherein transforming the signal quality classification information to the signal quality index comprises:
assigning a probability as a signal quality index, wherein the probability is associated with one of classes in the signal quality classification information.

12. The method of claim 1, wherein transforming the signal quality classification information to the signal quality index comprises:
assigning a weighted sum of probabilities as a signal quality index, wherein the probabilities are associated with classes in the signal quality classification information.

13. The method of claim 1, wherein the machine learning model comprises a neural network based model.

14. A vital sign monitoring device, comprising:
a buffer to store samples representative of a photoplethysmogram signals over a period of time that includes at least two cardiac cycles;
processor, when executing a set of instructions stored on non-transitory computer-readable medium, to:
process the samples;
extract features from the preprocessed samples;
apply a machine learning model using the features as input;
generate signal quality classification information based on the machine learning model;
extract vital sign information based on the samples if the signal quality classification information indicates sufficient quality; and
not extract vital sign information based on the samples if the signal quality classification information does not indicate sufficient quality; and
user interface output to output extracted vital sign information to a user.

15. The vital sign monitoring device of claim 14, wherein the processor is further to transform the signal quality classification information into a signal quality index.

16. The vital sign monitoring device of claim 15, wherein the user interface output is further to output the signal quality index or a derivation thereof.

17. A method for signal quality assessment, comprising:
extracting first features from first samples representative of a photo-plethysmograph signal over a period of time;
extracting second feature(s) from second samples representative of a motion sensor signal over the period of time;
extracting one or more cross-channel features from the first samples and the second samples, wherein the one or more cross-channel features include a sample correlation coefficient;
receiving, by a machine learning model, the first features, second features, and one or more cross-channel features, and outputting signal quality classification information based on the received features; and
generating a signal quality assessment based on the signal quality classification information.

18. The method of claim 17, wherein the one or more cross-channel features indicate a degree of dissimilarity between the first and second samples.

19. The method of claim 17, wherein the second feature(s) includes one or more of: variance, skewness, and kurtosis.

20. The method of claim 17, further comprising:
extracting third features from third samples representative of a further photo-plethysmograph signal over the period of time; and
extracting one or more further cross-channel features from the first samples and the third samples;
wherein the machine learning model is to further receive the third features and the one or more further cross-channel features.
